(19) United States
(12) Patent Application Publication
(10) Pub. No.: US 2026/0074022 A1
(43) Pub. Date: Mar. 12, 2026

(54) COMPUTERIZED SYSTEMS AND METHODS FOR DETECTING FEATURES IN ELECTRONIC IMAGES

(71) Applicant: Singular Genomics Systems, Inc., San Diego, CA (US)

(72) Inventors: Walter Pupin FU, San Diego, CA (US); Michael OUIMET, Del Mar, CA (US); Andrew WALTERS, San Diego, CA (US)

(21) Appl. No.: 19/326,544

(22) Filed: Sep. 11, 2025

Related U.S. Application Data
(60) Provisional application No. 63/693,905, filed on Sep. 12, 2024.

(51) Int. Cl. G16B 50/50; G06V 10/77; G06V 10/82; G16B 40/00

(52) U.S. Cl. CPC G16B 50/50; G06V 10/7715; G06V 10/82; G16B 40/00

(57) ABSTRACT

Disclosed herein, inter alia, are systems and methods for extracting features from sequencing images using machine learning, involving the construction of a kernel bank to handle variations in imaging conditions, compressing extracted vector intensities into super intensity vectors, and improving basecalling accuracy and robustness to misfocus and aberrations.

### CROSS-REFERENCES TO RELATED APPLICATIONS

This application claims the benefit of U.S. Provisional Application No. 63/693,905, filed Sep. 12, 2024, which is incorporated herein by reference in its entirety and for all purposes.

### BACKGROUND

The detection and quantification of bright features is a common problem in fluorescent microscopy. In applications such as next-generation sequencing (NGS), where maximizing the number of features imaged is crucial, the challenge is exacerbated by feature sizes and optics operating near the diffraction limit. Accurate sequencing relies on accurately quantifying the luminosity of a feature across various wavelength channels, a process known as extraction.

Disclosed herein, inter alia, are solutions to these and other problems in the art.

### BRIEF SUMMARY

In an aspect is provided a computer-implemented method for extracting features of sequencing images. In embodiments, the method includes: extracting vector intensities from an image patch of the sequencing images using at least a machine learning model, wherein the model is trained on a diverse set of sequencing images generated from a plurality of imaging systems to account for variations in imaging conditions and system parameters; and compressing the extracted vector intensities into a plurality of super intensity vectors, using at least the machine learning model, wherein the compression reduces dimensionality while preserving salient features of the sequencing images for downstream analysis.

In aspect is provided a non-transitory computer-readable medium storing instructions that, when executed by a processor, perform a method for analyzing a tissue sample.

### BRIEF DESCRIPTION OF THE DRAWINGS

FIG. 1. Schematic showing where the learned extraction network fits into the larger image processing pipeline. Inset: NA-based kernels. E: embedding dimension, discussed in the text.

FIGS. 2A-2C. Model displaying how varying the extraction kernel can optimize the signal-to-noise ratio of the extracted signal.

FIG. 3. Comparison of extraction and basecalling accuracy over cycles of an NGS experiment without the learned extraction network (V3 ERBC) and with it.

FIG. 4. Performance without/with the learned extraction network, evaluated on an imaging system that was not in the training set.

FIGS. 5A-5B. Performance of the learned extraction network evaluated using an NGS experiment where select cycles were deliberately misfocused. FIG. 5A: improvement in accuracy over the baseline (ERBC, no learned extraction) model. FIG. 5B: median Q-scores as a function of the applied misfocus. The dashed line marks the threshold for an example of poor performance, and the solid lines represented polynomial fits to the measured data.

FIG. 6. Performance of three basecaller architectures (ERBC, RTBC, Stateful) used without (dashed) and in conjunction with (solid) the learned extraction module.

FIG. 7. Basecalling accuracy with no learned extraction model (V3 ERBC), and with learned extraction models of different embedding dimension.

### DETAILED DESCRIPTION

The aspects and embodiments described herein relate to methods and systems which introduce a sophisticated machine learning-based framework to handle variations in feature shapes, using kernels based on the optical point-spread function and compressing extracted intensities into super-intensities for better basecalling accuracy.

### I. Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of. ” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid support (e.g. a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence. A “target sequence”, as used herein, refers to a sequence of a splint oligonucleotide that is the same, or substantially the same, as a sequence in a target polynucleotide (i.e., the target sequence of the splint oligonucleotide is the same, or substantially the same, as the target sequence in the target polynucleotide). In embodiments, the target sequence is a known sequence. In embodiments, the target sequence is selected from a set of known target sequences. In embodiments, the target sequence is located 5′ of the probe hybridization sequence of the target polynucleotide. A “subject sequence”, as used herein, refers to the sequence of interest in a target polynucleotide. For example, an oligonucleotide probe may be hybridized upstream of a subject sequence of a target polynucleotide and extending the oligonucleotide probe incorporates a sequence complementary to the subject sequence (i.e., a subject sequence complement) into the oligonucleotide probe. The extended oligonucleotide probe may then be processed further (e.g., circularized and/or amplified), and the subject sequence detected by, e.g., sequencing.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing.

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a microscope slide.

The term “spatial proximity” as used herein refers to a criterion or metric that groups cells based on their physical locations relative to each other. For example, cells that are geographically closer are more likely to be grouped together, suggesting that their spatial arrangement may reflect underlying biological or functional similarities. Spatial proximity may be reported as a value or vector indicating the relative distance between two or more cells. In embodiments, spatial proximity may be represented or quantified by creating a frequency vector, v_{SP}, where each element of the vector represents the distance between a cell and other cells.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality.

Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the claims.

The hardware and systems used to implement the various illustrative logics, logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of receiver smart objects, e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In embodiments, the functions of the systems described may be implemented in hardware, software, firmware, or any combination thereof. If implemented in software, the functions may be stored as one or more instructions or code on a non-transitory computer-readable storage medium or non-transitory processor-readable storage medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable storage media may include RAM, ROM, EEPROM, FLASH memory, CD-ROM or other optical disk storage, magnetic disk storage or other magnetic storage smart objects, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

The term “computing device” is used herein to refer to an electronic device equipped with at least a processor. Examples of computing devices may include system or device described herein, mobile devices (e.g., cellular telephones, wearable devices, smartphones, smartwatches, web-pads, tablet computers, Internet enabled cellular telephones, Wi-Fi® enabled electronic devices, personal data assistants (PDAs), laptop computers, etc.), personal computers, and server computing devices. In various embodiments, computing devices may be configured with memory and/or storage as well as networking capabilities, such as network transceiver(s) and antenna(s) configured to establish a wide area network (WAN) connection (e.g., a cellular network connection, etc.) and/or a local area network (LAN) connection (e.g., a wired/wireless connection to the Internet via a Wi-Fi® router, etc.). In embodiments, the computing device is a mobile device, such as a cellular telephone, wearable device, or smartphone (e.g., iPhone, Android, Blackberry, Palm, Symbian, or Windows).

As used in this application, the terms “component”, “module”, “system”, and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

As used herein, the term “feature” refers a site corresponding to a location on a solid support. A feature can contain only a single molecule or it can contain a population of several molecules of the same species (i.e., a cluster). Features of an array are typically discrete. The discrete features can be contiguous, or they can have spaces between each other. An “optically resolvable feature” refers to a feature capable of being distinguished from other features. Optics and sensor resolution has a finite limit as to a resolvable area. The Rayleigh criterion for the diffraction limit to resolution states that two images are just resolvable when the center of the diffraction pattern of one object is directly over the first minimum of the diffraction pattern of the other object. The minimal distance between two resolvable objects, r, is proportional to the wavelength of light and inversely proportional to the numerical aperture (NA). That is, the minimal distance between two resolvable objects is provided as r=0.61 wavelength/NA. If detecting light in the UV-vis spectrum (about 100 nm to about 900 nm), the remaining mutable variable to increase the resolution is the NA of the objective lens. A lens with a large NA will be able to resolve finer details. For example, a lens with larger NA is capable of detecting more light and so it produces a brighter image. Thus, a large NA lens provides more information to form a clear image, and so its resolving power will be higher. Typical dry objectives have an NA of about 0.80 to about 0.95. Higher NAs may be obtained by increasing the imaging medium refractive index between the object and the objective front lens for example immersing the lens in water (refractive index=1.33), glycerin (refractive index=1.47), or immersion oil (refractive index=1.51). Most oil immersion objectives have a maximum numerical aperture of 1.4, with the typical objectives having an NA ranging from 1.0 to 1.35.

As used herein, the term “super-intensity vector” refers to a vector-valued representation of a bright feature in a sequencing image, produced by compressing or aggregating a plurality of underlying intensity measurements. The underlying measurements may include pixel-level values, convolutional kernel outputs, or other intermediate descriptors extracted from an image patch. A super-intensity vector typically has a dimensionality equal to the number of wavelength channels, with each component corresponding to an optimally extracted fluorescence signal for a respective channel. In some embodiments, the components may further include derived quantities, such as normalized intensities, quality metrics, or latent variables produced by a machine-learning model. The purpose of the super-intensity vector is to preserve the salient spectral and spatial characteristics of the feature while reducing noise, crosstalk, and redundancy, thereby enabling robust downstream analyses such as basecalling, spot calling, or quality scoring.

As used herein, the term “image patch” refers to a defined sub-region of a sequencing image that contains at least one candidate bright feature. An image patch may be centered on a localized feature and encompass a window of pixels surrounding that feature, for example a square region of 9×9 pixels, although other sizes and shapes may be used depending on system resolution and feature density. The patch typically includes pixel data across multiple spectral channels and, in some implementations, across successive sequencing cycles. By analyzing patches rather than entire images, features can be processed largely independently, enabling efficient extraction of intensity values and reducing interference from distant regions of the image. In embodiments where the sample is arranged on a patterned array, the image patches may be aligned with the expected grid locations of features. In embodiments where the sample comprises randomly distributed features, patches may be dynamically defined around feature coordinates determined by a localization step.

As used herein, the term “wavelength channel” refers to an imaging channel corresponding to a defined spectral band in which fluorescence emissions are collected. In sequencing and other fluorescence-based assays, distinct fluorophores are typically assigned to different wavelength channels so that their emissions can be separated by the optical system. The number of wavelength channels may vary depending on the experimental design. In some embodiments, the system employs two channels, for example when two-color chemistry is used and each base is identified from combinations of two dyes. In other embodiments, the system employs three channels, for instance where three spectrally distinct dyes are sufficient to encode the required information. In further embodiments, the system employs four channels, such as in sequencing-by-synthesis platforms that assign a unique dye to each of the four nucleotides. Regardless of the specific number of channels, the methods described herein extract and compress vector intensities from each channel of an image patch and generate corresponding super-intensity vectors that preserve the salient signal characteristics. For example, in one embodiment the system employs four distinct wavelength channels spanning the visible spectrum. A first channel is centered in the blue region, such as near 470 nm, suitable for dyes like Alexa Fluor® 488 or Atto 488. A second channel is centered in the green-yellow region, such as near 550 nm, capturing emissions from dyes such as Cy3 or Alexa Fluor® 546. A third channel is centered in the orange-red region, such as near 600 nm, compatible with dyes such as Texas Red or Alexa Fluor® 594. A fourth channel is centered in the far-red region, such as near 670 nm, accommodating dyes such as Cy5 or Alexa Fluor® 647. Each channel thus corresponds to a distinct fluorophore emission band within the visible spectrum, and the combination of four channels enables simultaneous discrimination of four different base-associated signals during sequencing.

As used herein, the term “assumed optical numerical aperture” refers to a numerical aperture value that is selected or estimated in order to construct a kernel that mimics the expected point-spread function of an imaging system. The numerical aperture of a microscope objective determines the size and shape of the diffraction-limited spot formed by a point emitter, and therefore influences the spatial distribution of light across the pixels of the image sensor. In practice, an imaging system may operate at a particular NA, but the actual effective NA can vary depending on optical aberrations, refractive index mismatches, or changes in focus. To account for these possibilities, kernels may be constructed using a range of assumed NA values (e.g., 0.4, 0.6, 0.8, or 1.0) even if the physical system nominally operates at a single NA. By convolving image patches with kernels derived from multiple assumed NA values, the extraction method generates intensity responses that capture how a feature would appear under different focus or optical conditions. In embodiments, “assumed” NA implies that it is not necessarily the physical NA, but a design parameter used in kernel construction.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Systems & Devices

In an aspect is provided a non-transitory computer-readable medium storing instructions that, when executed by a processor, perform a method for analyzing a tissue sample, the method including: extracting vector intensities, using at least a machine learning model trained on a set of sequencing images generated from a plurality of imaging systems, of an image patch of the sequencing images; and compressing, using at least the machine learning model, the vector intensities into a plurality of super intensity vectors.

In another aspect is provided a non-transitory computer-readable medium storing instructions that, when executed by one or more processors, cause the processors to perform a method for analyzing sequencing images of a biological sample, the method including: receiving a sequencing image of the biological sample acquired by any of a plurality of imaging systems; selecting an image patch from the sequencing image; extracting, using a trained machine-learning model, a plurality of pixel-intensity vectors from the image patch, each pixel-intensity vector encoding intensity values for a corresponding pixel or local neighborhood across one or more channels and/or cycles; and compressing, using the machine-learning model, the plurality of pixel-intensity vectors into a plurality of super-intensity vectors by aggregating pixel-intensity vectors assigned to respective bright features, wherein a super-intensity vector is a vector-valued representation of a corresponding bright feature within the image patch. In embodiments, the machine-learning model is trained on training data comprising sequencing images acquired from a plurality of different imaging systems to learn instrument-invariant feature representations.

The disclosed extraction approach is equally applicable to sequencing performed on standard immobilized oligonucleotide arrays, such as those used in next-generation sequencing platforms, and/or tissue or cell-based samples. In these implementations, the fluorescent features arise from clonally amplified clusters or from single immobilized molecules distributed across a flow cell surface. The same fundamental challenge exists: the accurate quantification of fluorescence intensity for each feature across multiple spectral channels, with the added complication that the features may be closely spaced on a regular grid or on a randomly distributed array of beads. The convolution with a bank of kernels, followed by compression through a machine learning network, allows the system to robustly extract feature intensities even under conditions of optical aberration, crosstalk, or defocus. In the case of patterned arrays, the kernel bank may be constructed with reference to the underlying grid, so that neighboring features are properly accounted for during extraction. In the case of random arrays, feature localization precedes kernel application, and the extracted pixel-intensity vectors are generated from image patches centered on the localized features. In either context, the resulting super-intensity vectors provide accurate representations of the feature intensities suitable for downstream basecalling. Because the learned extraction network is trained on data spanning multiple instruments and focus conditions, it generalizes not only to tissue-based fluorescence assays but also to high-throughput array-based sequencing workflows, thereby broadening the utility of the method to both spatial sequencing and conventional SBS systems.

In embodiments, the non-transitory computer-readable medium is a computing device. In embodiments, the computing device is a personal computer system, server computer system, hand-held or laptop device, multiprocessor system, microprocessor-based system, set top box, programmable consumer electronic, network PC, minicomputer system, mainframe computer system, smartphone, or distributed cloud computing environments that include any of the above systems or devices. The computing device can include one or more processors or processing units, a memory architecture that may include RAM and non-volatile memory. The memory architecture may further include removable/non-removable, volatile/non-volatile computer system storage media. Further, the memory architecture may include one or more readers for reading from and writing to a non-removable, non-volatile magnetic media, such as a hard drive, a magnetic disk drive for reading from and writing to a removable, non-volatile magnetic disk, and/or an optical disk drive for reading from or writing to a removable, non-volatile optical disk such as a CD-ROM or DVD-ROM.

In another aspect is provided a system for analyzing a tissue sample, the system including: (a) a memory storing instructions; (b) a processor configured to execute the instructions to: extracting vector intensities, using at least a machine learning model trained on a set of sequencing images generated from a plurality of imaging systems, of an image patch of the sequencing images; and compressing, using at least the machine learning model, the vector intensities into a plurality of super intensity. Around each bright feature candidate, the processor defines an image patch and extracts a set of vector intensities using a machine-learning model trained on images obtained from multiple imaging systems. The model operates on convolved pixel data derived from a kernel bank, producing vector representations that capture intensity values across channels and cycles. These vector intensities are then compressed by the model into a reduced set of super-intensities, each corresponding to an optimally extracted signal for a respective spectral channel. The super-intensities provide robust feature quantification even in the presence of optical aberrations, focus variations, or instrument-specific distortions, and they are suitable for direct input into a downstream basecalling algorithm.

In embodiments, the system includes one or more processing units CPU(s) (also referred to as processors), one or more network interfaces, a user interface including a display and an input module, a non-persistent, a persistent memory, and one or more communication buses for interconnecting these components. The one or more communication buses optionally include circuitry (sometimes called a chipset) that interconnects and controls communications between system components. The non-persistent memory typically includes high-speed random access memory, such as DRAM, SRAM, DDR RAM, ROM, EEPROM, flash memory, whereas the persistent memory typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory optionally includes one or more storage devices remotely located from the CPU(s). The persistent memory, and the non-volatile memory device(s) within the non-persistent memory, comprise non-transitory computer readable storage medium. In embodiments, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations.

In embodiments, the computing device includes memory in electronic communication with the processor. The memory architecture may include at least one program module implemented as executable instructions that are configured to carry out one or more steps of a method set forth herein. For example, executable instructions may include an operating system, one or more application programs, other program modules, and program data. Generally, program modules may include routines, programs, objects, components, logic, and data structures that perform particular tasks. A computing device can optionally communicate with one or more external devices such as a keyboard, a pointing device (e.g., a mouse), a display, such as a graphical user interface (GUI), or other device that facilitates interaction of a use with the unmanned autonomous vehicle. Similarly, the computing device can communicate with other devices (e.g., via network card, modem, etc.). Such communication can occur via I/O interfaces. In embodiments, the computing system may communicate with one or more networks such as a local area network (LAN), a general wide area network (WAN), and/or a public network (e.g., the Internet) via a suitable network adapter.

In embodiments, the systems and devices generate data that may be used to form an image. In embodiments, the image includes a 2D or 3D representation of the tissue. In some embodiments, one or more of the images include an image of other analytes, such as proteins in a biological sample. In some embodiments, an image is acquired using transmission light microscopy (e.g., bright field transmission light microscopy, dark field transmission light microscopy, oblique illumination transmission light microscopy, dispersion staining transmission light microscopy, phase contrast transmission light microscopy, differential interference contrast transmission light microscopy, emission imaging, etc.). In embodiments, the image is in any file format including but not limited to JPEG/JFIF, TIFF, Exif, PDF, EPS, GIF, BMP, PNG, PPM, PGM, PBM, PNM, WebP, HDR raster formats, HEIF, BAT, BPG, DEEP, DRW, ECW, FITS, FLIF, ICO, ILBM, IMG, PAM, PCX, PGF, JPEG XR, Layered Image File Format, PLBM, SGI, SID, CDS, CPT, PSD, PSP, XCF, PDN, CGM, SVG, PostScript, PCT, WMF, EMF, SWF, XAML, and/or RAW. In embodiments, the image is represented as an array (e.g., matrix) comprising a plurality of pixels, such that the location of each respective pixel in the plurality of pixels in the array (e.g., matrix) corresponds to its original location in the image. In some embodiments, an image is represented as a vector comprising a plurality of pixels, such that each respective pixel in the plurality of pixels in the vector comprises spatial information corresponding to its original location in the image.

In embodiments, a pixel includes one or more pixel values (e.g., intensity value). In embodiments, each respective pixel in the plurality of pixels includes one pixel intensity value, such that the plurality of pixels represents a single-channel image comprising a one-dimensional integer vector comprising the respective pixel values for each respective pixel. For example, an 8-bit single-channel image (e.g., grey-scale) can include 2⁸ or 256 different pixel values (e.g., 0-255). In embodiments, each respective pixel in the plurality of pixels of an image includes a plurality of pixel values, such that the plurality of pixels represents a multi-channel image comprising a multi-dimensional integer vector, where each vector element represents a plurality of pixel values for each respective pixel. For example, a 24-bit 3-channel image (e.g., RGB color) can include 2²⁴ (e.g., 2^{8×3}) different pixel values, where each vector element comprises 3 components, each between 0-255. In some embodiments, an n-bit image includes up to 2ⁿ different pixel values, where n is any positive integer.

In embodiments, each pixel in the plurality of pixels of the image has a pixel size (resolution) between 0.8 pm and 4.0 pm. In embodiments the pixel size is derived by dividing the camera pixel size (resolution) by the magnification of the objective lens of the camera used to capture values for the plurality of pixels. In embodiments, each pixel in the plurality of pixels has a pixel size between 0.4 pm and 5.0 pm. In embodiments, each pixel in the plurality of pixels of the image has a pixel size (resolution) between 0.8 pm and 4.0 pm or between 0.4 pm and 5.0 pm.

In embodiments, the data processor provides the image for display via a display of the computing device. In embodiments, the image is provided for display via a GUI configured within the display of the computing device. In embodiments, the data processor receives an input identifying one or more modifications and/or one or more image analysis steps based on the provided image. For example, the display of the computing device can include a touchscreen display configured to receive a user input identifying a respective pattern of an image of the biological sample on the displayed image. In embodiments, the GUI can be configured to receive a user provided input identifying the modifications and/or one or more image analysis steps. The GUI may also be configured to accept other forms of user input, such as cursor-based selections, typed commands, or menu-driven choices, thereby enabling the user to guide subsequent image analysis operations performed by the system.

### III. Methods

In an aspect is provided a computer-implemented method for extracting features of sequencing images. In embodiments, the method includes extracting vector intensities, using at least a machine learning model (e.g., a machine learning model trained on a set of sequencing images generated from a plurality of imaging systems), of an image patch of the sequencing images; and compressing, using at least the machine learning model, the vector intensities into a plurality of super intensity vectors.

A kernel may refer to a small matrix (also called a filter or convolution matrix) that is computationally applied to an image or data to extract specific features, such as edges, textures, or patterns. The kernel is “slid” over the input data, performing a mathematical operation (typically convolution) to generate a transformed output. A common operation is convolution, where each element of the kernel is multiplied by the corresponding elements of the image or data as the kernel moves across it. The results are then summed to produce a single output value for each location. In embodiments, the kernels mimic the expected optical point-spread function (PSF), helping to extract intensity values that correspond to the features (such as bright spots) in the sequencing images. In embodiments, the dynamic kernels are constructed to account for system properties like the numerical aperture (NA) and/or diffraction limits.

The term sequencing may refer to methods for determining a primary structure or sequence of a polynucleotide such as a nucleic acid including DNA or RNA. More specifically, DNA sequencing is the process of determining an order of nucleotide bases (adenine, guanine, cytosine and thymine) in a given DNA fragment. Such sequencing methods commonly include calling a base at a position in a nucleic acid, where the called base is used to determine a sequence for the nucleic acid. An intensity value (e.g., a fluorescence signal) corresponding to a base that is incorporated into a nucleic acid at a particular position can indicate the base at that position. For example, four different types of fluorescence may be used, corresponding to the four types of bases to be identified. The nucleic acids are amenable to relatively inexpensive and efficient imaging techniques in which the nucleic acids are captured in four color images, one for each type of fluorescence used. The four images can then be processed through software to extract intensity information. Examples of incorporation are sequencing by synthesis, sequencing by ligation, and sequencing by hybridization. The detected signals (e.g., fluorescent signal emissions) can be used to call a base at a position of the nucleic acid, i.e., perform basecalling. The intensity value for a target nucleic acid template can correspond to one pixel or multiple pixels of an image, or there can be multiple templates for a pixel (i.e., more than one template per pixel). Regardless, an intensity value for each of the four bases can be assigned to a template. Naively, one can call the base corresponding to the maximum intensity value, but this has a high error rate. For example, the determination of the intensity value can be incorrect due to optical effects (e.g., overlap in spectrum of the various intensity signals) and spatial effects (e.g., when multiple templates correspond to a single pixel).

Various sequencing techniques can be employed to obtain intensity values from nucleic acid molecules. For example, nucleic acid molecules may be deposited on a substrate, such as a slide, in an ordered array (e.g., a nanopatterned lattice) or in a non-ordered (e.g., random) fashion. The ordered arrays may follow a specific pattern, such as a rectangular/square lattice, a checkerboard configuration (where lattice positions neighbor at corners but not sides), or a hexagonal lattice. Each distinct location on the substrate corresponds to a unique template molecule. In other embodiments, sequencing is performed as molecules flow through channels within the substrate. During the sequencing process, intensity values from molecules on the substrate are captured simultaneously for a given cycle, with each cycle corresponding to a different nucleotide position on the molecule. For instance, an image of the substrate may capture various locations emitting light, where each location emits signals at different wavelengths, corresponding to distinct nucleotide bases. Each image represents a specific cycle of the sequencing process, and the intensity values obtained can be used to identify the nucleotide base at each position of the nucleic acid molecules.

In another aspect a computer-implemented method for extracting features of sequencing images is provided. In embodiments, the method includes: extracting vector intensities from an image patch of the sequencing images using at least a machine learning model, wherein the model is trained on a diverse set of sequencing images generated from a plurality of imaging systems to account for variations in imaging conditions and system parameters; and compressing the extracted vector intensities into a plurality of super intensity vectors, using at least the machine learning model, wherein the compression reduces dimensionality while preserving salient features of the sequencing images for downstream analysis.

In embodiments, the method involves selecting an image patch surrounding a bright feature candidate and generating a plurality of vector intensities from that patch using a machine-learning model. The model is trained on a diverse collection of sequencing images obtained from multiple imaging systems, such as widefield fluorescence, confocal, or time-delay integration scanners, so that the model learns to accommodate variations in optical aberrations, illumination profiles, noise characteristics, and focus quality. By leveraging training data from multiple platforms, the model produces feature representations that are robust to system-specific distortions and generalizable to new instruments. The extracted vector intensities may be derived from pixel values processed through a bank of kernels designed to mimic point-spread functions, thereby capturing the spatial distribution of light around each bright feature. These vectors are then compressed by the machine-learning model into a smaller set of super-intensity vectors, with each super-intensity vector corresponding to a respective spectral channel. The compression process reduces dimensionality while retaining the salient features of the signal, allowing downstream modules, such as basecallers, spot-callers, or quality-score generators, to operate on a simplified but information-rich representation. In this way, the method improves accuracy under conditions of crosstalk, background contamination, or optical misfocus, while at the same time enabling computational efficiency by distilling high-dimensional data into compact, discriminative feature vectors.

In embodiments, the method includes: extracting vector intensities from an image patch of the sequencing images using at least a machine learning model, wherein the model is trained on a diverse set of sequencing images generated from a plurality of imaging systems to account for variations in imaging conditions, such as optical aberrations, misfocus, and system parameters; and compressing the extracted vector intensities into a plurality of super intensity vectors using at least the machine learning model, wherein the compression not only reduces dimensionality but also enhances robustness by preserving the salient features of the sequencing images, enabling improved accuracy in downstream analysis, particularly in the presence of defocused or aberrant images.

In embodiments, the method further includes inputting the super intensity vectors into a base caller. In embodiments, the method further includes inputting the super intensity vectors into a basecaller, wherein the basecaller processes the super intensity vectors to determine nucleotide sequences, independent of the internal structure or training of the machine learning model used for feature extraction.

In embodiments, the sequencing images of the image patch are aligned across successive cycles and spectral channels. In embodiments, the sequencing images corresponding to a given image patch are aligned across successive cycles and spectral channels to ensure that the same physical feature is consistently represented in each frame. Such alignment can be performed with subpixel precision and may involve registration methods such as cross-correlation, affine transformations, or non-linear warping to compensate for mechanical drift or optical distortions between cycles. By aligning the images, the system ensures that pixel-level intensity values extracted from the patch correspond to the same underlying bright feature over time and across wavelengths. This alignment reduces artifacts caused by cycle-to-cycle motion, chromatic aberrations, or local deformations, thereby improving the accuracy of the vector intensities extracted from the patch. The resulting aligned data provide a consistent basis for subsequent compression into super-intensity vectors, which rely on accurate cross-cycle and cross-channel correspondence to preserve the true spectral signature of the feature.

In embodiments, the image patch is arranged around each extracted feature. In embodiments, the image patch is centered on each extracted feature. In embodiments, the image patch encompasses each extracted feature. In embodiments, the image patch surrounds each extracted feature in a predetermined manner.

In embodiments, the plurality of super intensity vector represents optimally-extracted signals of a wavelength channel of the image patch. In embodiments, the plurality of super intensity vectors represents optimally-extracted signals corresponding to a wavelength channel of the image patch. In embodiments, the plurality of super intensity vectors represents signals corresponding to a wavelength channel of the image patch, optimized based on signal-to-noise ratio across the image patch. In embodiments, the plurality of super intensity vectors represents signals corresponding to a wavelength channel of the image patch, extracted and optimized using a machine learning model trained on sequencing images. In embodiments, the plurality of super intensity vectors represents signals corresponding to a wavelength channel of the image patch, optimally-extracted through spatial and spectral alignment across multiple cycles and channels. In embodiments, the plurality of super intensity vectors represents signals corresponding to a wavelength channel of the image patch, optimally-extracted to enhance feature detection and minimize background interference.

In embodiments, the method further includes generating the plurality of super intensity vectors for each wavelength channel by convolving the aligned image patches with a bank of kernels. In embodiments, each kernel mimics an expected optical point-spread function. In embodiments, each kernel is designed to mimic an expected optical point-spread function (PSF) corresponding to the imaging system. In embodiments, each kernel is designed to mimic an expected optical point-spread function (PSF) for each wavelength channel, adjusted according to the optical properties and imaging conditions specific to that channel. In embodiments, each kernel is designed to dynamically mimic an expected optical point-spread function (PSF) that evolves based on temporal variations or focus drift during the sequencing process.

In embodiments, the method further includes constructing a different kernel bank for each wavelength channel, wherein each different kernel bank corresponds to a predominant wavelength of the wavelength channel; and expanding the different kernel banks to include copies with different subpixel translations based on a previously calculated feature location. In embodiments, the method includes constructing a distinct kernel bank for each wavelength channel, wherein each kernel bank is tuned to a predominant wavelength within the respective wavelength channel; and expanding the kernel banks to include variants with different subpixel translations, wherein the translations are based on previously calculated feature locations to account for subpixel-level shifts in feature alignment.

In embodiments, each of the kernels are constructed based on an assumed optical numerical aperture. In embodiments, the assumed optical numerical aperture is at least one of 0.4, 0.6, 0.8, and 1.0. In embodiments, the assumed optical numerical aperture is 0.4. In embodiments, the assumed optical numerical aperture is 0.6. In embodiments, the assumed optical numerical aperture is 0.8. In embodiments, the assumed optical numerical aperture is 1.0. In embodiments, each kernel is constructed based on an assumed optical numerical aperture (NA) of the imaging system, with the numerical aperture determining the spatial resolution and the shape of the point-spread function (PSF) modeled in the kernel.

In embodiments, the method includes creating a high-resolution Airy disk for each numerical aperture thereby creating a series of Airy disks of different widths. In embodiments, the method includes surrounding each Airy disk by a hexagonal ring. In embodiments, the method includes creating a high-resolution Airy disk for each assumed numerical aperture (NA), thereby generating a series of Airy disks with varying widths corresponding to the different numerical apertures, each disk representing the diffraction pattern produced by the optical system.

In embodiments, kernels are derived from high-resolution Airy disk models generated for a set of assumed numerical apertures, thereby producing a family of disks of differing width that reflect diffraction behavior across focus conditions. The high-resolution kernels can be downsampled to the sensor pixel pitch after optional apodization or band-limiting, and a pseudo-inverse may be computed to temper ringing and to stabilize subsequent convolution on finite patches. In implementations where features are disposed on a patterned substrate, the Airy-derived central lobe may be augmented with a surrounding arrangement of opposite-sign elements positioned at the expected locations of nearest neighbors so as to suppress crosstalk. For substrates patterned on a hexagonal lattice, these opposite-sign elements can be placed on a hexagonal ring at a radius corresponding to the lattice spacing, thereby attenuating leakage from the six immediate neighbors while preserving the target feature's central energy.

In embodiments, the spatial arrangement of opposite-sign elements is matched to the underlying array geometry. For square or rectangular lattices, the surrounding elements can be positioned on a square ring aligned to the lattice vectors; for triangular lattices, on a triangular ring; and for quasi-periodic or multi-pitch patterns, on a composite ring formed from the superposition of the relevant neighbor offsets. The ring may be extended to multiple radii to address second-and third-neighbor contributions, with weights that decay with distance or are optimized empirically to balance signal retention and neighbor suppression. Where features are not arranged on a fixed grid, as in bead-based or randomly distributed arrays, the kernel can incorporate a parametric “neighbor mask” that places opposite-sign lobes at offsets predicted from a localization step (e.g., k-nearest neighbors within the patch), enabling adaptive suppression at run time without requiring a global lattice model. Channel-specific and anisotropic effects can also be accommodated. For example, the central lobe can be elongated to approximate astigmatic PSFs or tilted to account for residual scan skew, and the surrounding suppression elements can be shaped or weighted differently per wavelength channel to reflect chromatic aberrations. In some embodiments, the Airy-based kernels are replaced or complemented by Gaussian, difference-of-Gaussian, or Laplacian-of-Gaussian approximations that are computationally lighter while still capturing the essential spatial spread; these may likewise be paired with geometry-aware suppression masks. In other embodiments, the kernel bank is constructed as linear combinations of a complete optical basis, such as Zernike polynomials, with coefficients learned during training so that both the central lobe and any neighbor-suppression structure emerge from data while remaining physically plausible. Subpixel translations of each kernel variant may be precomputed so that, after patch alignment, the kernel instance best matching the feature's subpixel centroid can be selected. Across these embodiments, the resulting kernel bank yields a set of scalar responses per channel that encode how a candidate feature would appear under different assumed optical conditions and neighborhood configurations; these responses form the vector intensities that are subsequently compressed by the machine-learning model into super-intensity vectors.

In embodiments, the method includes calculating a pseudo-inverse of a pattern created by each Airy disk; and downsampling the pseudo-inverse to a true pixel pitch, resulting in a kernel that mimics a point spread function with depressions for nearest neighbor crosstalk suppression. In embodiments, the method further includes: calculating a pseudo-inverse of a pattern generated by each Airy disk; and downsampling the pseudo-inverse to match the true pixel pitch of the imaging system, thereby producing a kernel that mimics a point-spread function (PSF) with depressions designed to suppress nearest neighbor crosstalk. In embodiments, the method further includes: calculating a pseudo-inverse of a diffraction pattern generated by each high-resolution Airy disk, wherein the Airy disk corresponds to the numerical aperture of the imaging system; and downsampling the pseudo-inverse to match the true pixel pitch of the imaging system, ensuring that the resulting kernel mimics the point-spread function (PSF) with strategically positioned depressions to mitigate nearest neighbor crosstalk and improve signal isolation between adjacent pixels.

In embodiments, the compressing the extracted vector intensities includes: projecting, using at least a linear transformation, the extracted vector intensities of the image patch into an embedding; and passing the embedding through a plurality of activation functions and a plurality of linear transformations into the plurality of super intensity vectors. In embodiments, the compressing of the extracted vector intensities includes: projecting the extracted vector intensities of the image patch into an embedding space using at least one linear transformation, wherein the embedding captures the key features of the image patch in a lower-dimensional space; and passing the embedding through a sequence of activation functions and linear transformations, wherein each transformation and activation function progressively refines the representation, resulting in the plurality of super intensity vectors that capture the compressed, salient features of the image patch.

In embodiments, embedding refers to a vector representation of the extracted intensities of an image patch in a transformed space produced by one or more linear or non-linear mappings. In some embodiments, the extracted vector intensities are projected by a linear transformation into an embedding space of dimension E. The embedding captures salient relationships among the intensity values, such as correlations across channels, cycles, or kernels, in a form that is more amenable to subsequent processing by the machine-learning model. The embedding may then be passed through one or more activation functions and additional linear transformations, ultimately yielding the super-intensity vectors. The dimensionality E may vary depending on implementation, for example between 16 and 256, and need not equal the number of input or output channels.

In embodiments, the method further includes analyzing, using at least the machine learning model, each extracted feature of the image patch independent of other features of the image patch. In embodiments, the method further includes: analyzing, using at least the machine learning model, each extracted feature of the image patch independently from other features, wherein the model isolates and evaluates each feature in a manner that prevents interference or cross-correlation with adjacent features, ensuring that each feature is assessed in a context-specific manner.

In another aspect is provided a computer-implemented method for extracting features of an image, including: constructing a kernel bank including learned linear combinations of a set of precomputed two-dimensional basis vectors, such as polynomials, wavelets, or pseudorandom patterns trained simultaneously with a machine learning nonlinear extraction network that is trained on a set of sequencing images generated from a plurality of imaging systems; and pre-computing, using at least the machine learning nonlinear extraction network, a set of optimal linear combinations.

In embodiments, the method includes constructing a kernel bank consisting of learned linear combinations of a set of precomputed two-dimensional basis vectors, such as polynomials, wavelets, or pseudorandom patterns, wherein the kernel bank is trained simultaneously with a machine learning nonlinear extraction network, the network being trained on a set of sequencing images generated from a plurality of imaging systems; and precomputing, using at least the machine learning nonlinear extraction network, a set of optimal linear combinations designed to enhance feature extraction from the sequencing images.

In embodiments, the method includes constructing a kernel bank consisting of learned linear combinations of a set of precomputed two-dimensional basis vectors, such as polynomials, wavelets, or pseudorandom patterns, wherein the kernel bank is trained simultaneously with a machine learning nonlinear extraction network, the network being trained on a set of images generated from multiple channels, which may correspond to different wavelengths, measurement devices, viewing angles, or imaging modalities; and precomputing, using at least the machine learning nonlinear extraction network, a set of optimal linear combinations designed to enhance feature extraction across the multiple channels.

Methods as described herein can be implemented by way of machine (e.g., computer processor) executable code stored on an electronic storage location of the computer system, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre compiled or as-compiled fashion.

Examples of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media (e.g., computer-readable media) include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for tissue sample analysis. Examples of UIs include, without limitation, a graphical user interface (GUI) and web-based user interface. Methods and systems of the present disclosure can be implemented by way of one or more algorithms. An algorithm can be implemented by way of software upon execution by the central processing unit.

According to certain embodiments, the above-described data feeds may be stored in databases such as database servers that store master data as well as logging and trace information. The databases may also provide an API and/or API access (e.g., for open source) to the web server for data interchange based on JSON specifications. According to certain embodiments, the database servers may be optimally designed for storing large amounts of data, responding quickly to incoming requests, having a high availability and historizing master data.

The system contemplates uses in association with web services, utility computing, pervasive and individualized computing, security and identity solutions, autonomic computing, cloud computing, commodity computing, mobility and wireless solutions, open source, biometrics, grid computing, and/or mesh computing.

Any databases discussed herein may include relational, hierarchical, graphical, blockchain, object-oriented structure, and/or any other database configurations. Any database may also include a flat file structure wherein data may be stored in a single file in the form of rows and columns, with no structure for indexing and no structural relationships between records. For example, a flat file structure may include a delimited text file, a CSV (comma-separated values) file, and/or any other suitable flat file structure. Common database products that may be used to implement the databases include DB2® by IBM® (Armonk, NY), various database products available from ORACLE® Corporation (Redwood Shores, CA), MICROSOFT ACCESS® or MICROSOFT SQL SERVER® by MICROSOFT® Corporation (Redmond, Washington), MYSQL® by MySQL AB (Uppsala, Sweden), MONGODB®, Redis, Apache Cassandra®, HBASE® by APACHE®, MapR-DB by the MAPR® corporation, or any other suitable database product. Moreover, any database may be organized in any suitable manner, for example, as data tables or lookup tables. Each record may be a single file, a series of files, a linked series of data fields, or any other data structure.

As used herein, big data may refer to partially or fully structured, semi-structured, or unstructured data sets including millions of rows and hundreds of thousands of columns. A big data set may be compiled, for example, from a history of purchase transactions over time, from web registrations, from social media, from records of charge (ROC), from summaries of charges (SOC), from internal data, or from other suitable sources. Big data sets may be compiled without descriptive metadata such as column types, counts, percentiles, or other interpretive-aid data points.

Association of certain data may be accomplished through any desired data association technique such as those known or practiced in the art. For example, the association may be accomplished either manually or automatically. Automatic association techniques may include, for example, a database search, a database merge, GREP, AGREP, SQL, using a key field in the tables to speed searches, sequential searches through all the tables and files, sorting records in the file according to a known order to simplify lookup, and/or the like. The association step may be accomplished by a database merge function, for example, using a “key field” in pre-selected databases or data sectors. Various database tuning steps are contemplated to optimize database performance. For example, frequently used files such as indexes may be placed on separate file systems to reduce In/Out (“I/O”) bottlenecks.

More particularly, a “key field” partitions the database according to the high-level class of objects defined by the key field. For example, certain types of data may be designated as a key field in a plurality of related data tables and the data tables may then be linked on the basis of the type of data in the key field. The data corresponding to the key field in each of the linked data tables is preferably the same or of the same type. However, data tables having similar, though not identical, data in the key fields may also be linked by using AGREP, for example. In accordance with one embodiment, any suitable data storage technique may be utilized to store data without a standard format. Data sets may be stored using any suitable technique, including, for example, storing individual files using an ISO/IEC 7816-4 file structure; implementing a domain whereby a dedicated file is selected that exposes one or more elementary files containing one or more data sets; using data sets stored in individual files using a hierarchical filing system; data sets stored as records in a single file (including compression, SQL accessible, hashed via one or more keys, numeric, alphabetical by first tuple, etc.); data stored as Binary Large Object (BLOB); data stored as ungrouped data elements encoded using ISO/IEC 7816-6 data elements; data stored as ungrouped data elements encoded using ISO/IEC Abstract Syntax Notation (ASN.1) as in ISO/IEC 8824 and 8825; other proprietary techniques that may include fractal compression methods, image compression methods, etc.

In various embodiments, the ability to store a wide variety of information in different formats is facilitated by storing the information as a BLOB. Thus, any binary information can be stored in a storage space associated with a data set. As discussed above, the binary information may be stored in association with the system or external to but affiliated with the system. The BLOB method may store data sets as ungrouped data elements formatted as a block of binary via a fixed memory offset using either fixed storage allocation, circular queue techniques, or best practices with respect to memory management (e.g., paged memory, least recently used, etc.). By using BLOB methods, the ability to store various data sets that have different formats facilitates the storage of data, in the database or associated with the system, by multiple and unrelated owners of the data sets. For example, a first data set which may be stored may be provided by a first party, a second data set which may be stored may be provided by an unrelated second party, and yet a third data set which may be stored may be provided by a third party unrelated to the first and second party. Each of these three exemplary data sets may contain different information that is stored using different data storage formats and/or techniques. Further, each data set may contain subsets of data that also may be distinct from other subsets.

As stated above, in various embodiments, the data can be stored without regard to a common format. However, the data set (e.g., BLOB) may be annotated in a standard manner when provided for manipulating the data in the database or system. The annotation may comprise a short header, trailer, or other appropriate indicator related to each data set that is configured to convey information useful in managing the various data sets. For example, the annotation may be called a “condition header,” “header,” “trailer,” or “status,” herein, and may comprise an indication of the status of the data set or may include an identifier correlated to a specific issuer or owner of the data. In one example, the first three bytes of each data set BLOB may be configured or configurable to indicate the status of that particular data set; e.g., LOADED, INITIALIZED, READY, BLOCKED, REMOVABLE, or DELETED. Subsequent bytes of data may be used to indicate for example, the identity of the issuer, user, transaction/membership account identifier or the like. Each of these condition annotations are further discussed herein.

The data set annotation may also be used for other types of status information as well as various other purposes. For example, the data set annotation may include security information establishing access levels. The access levels may, for example, be configured to permit only certain individuals, levels of employees, companies, or other entities to access data sets, or to permit access to specific data sets based on the transaction, merchant, issuer, user, or the like. Furthermore, the security information may restrict/permit only certain actions, such as accessing, modifying, and/or deleting data sets. In one example, the data set annotation indicates that only the data set owner or the user are permitted to delete a data set, various identified users may be permitted to access the data set for reading, and others are altogether excluded from accessing the data set. However, other access restriction parameters may also be used allowing various entities to access a data set with various permission levels as appropriate.

The data, including the header or trailer, may be received by a standalone interaction device configured to add, delete, modify, or augment the data in accordance with the header or trailer. As such, in one embodiment, the header or trailer is not stored on the transaction device along with the associated issuer-owned data, but instead the appropriate action may be taken by providing to the user, at the standalone device, the appropriate option for the action to be taken. The system may contemplate a data storage arrangement wherein the header or trailer, or header or trailer history, of the data is stored on the system, device or transaction instrument in relation to the appropriate data.

One skilled in the art will also appreciate that, for security reasons, any databases, systems, devices, servers, or other components of the system may consist of any combination thereof at a single location or at multiple locations, wherein each database or system includes any of various suitable security features, such as firewalls, access codes, encryption, decryption, compression, decompression, and/or the like.

Practitioners will also appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page such as, for example, free text entry using a keyboard, selection of menu items, check boxes, option boxes, and the like.

The data may be big data that is processed by a distributed computing cluster. The distributed computing cluster may be, for example, a HADOOP® software cluster configured to process and store big data sets with some of nodes comprising a distributed storage system and some of nodes comprising a distributed processing system. In that regard, distributed computing cluster may be configured to support a HADOOP® software distributed file system (HDFS) as specified by the Apache Software Foundation at www.hadoop.apache.org/docs.

As used herein, the term “network” includes any cloud, cloud computing system, or electronic communications system or method which incorporates hardware and/or software components. Communication among the parties may be accomplished through any suitable communication channels, such as, for example, a telephone network, an extranet, an intranet, internet, point of interaction device (point of sale device, personal digital assistant (e.g., an IPHONE® device, a BLACKBERRY® device), cellular phone, kiosk, etc.), online communications, satellite communications, off-line communications, wireless communications, transponder communications, local area network (LAN), wide area network (WAN), virtual private network (VPN), networked or linked devices, keyboard, mouse, and/or any suitable communication or data input modality. Moreover, although the system is frequently described herein as being implemented with TCP/IP communications protocols, the system may also be implemented using IPX, APPLETALK® program, IP-6, NetBIOS, OSI, any tunneling protocol (e.g., IPsec, SSH, etc.), or any number of existing or future protocols. If the network is in the nature of a public network, such as the internet, it may be advantageous to presume the network to be insecure and open to eavesdroppers. Specific information related to the protocols, standards, and application software utilized in connection with the internet is generally known to those skilled in the art and, as such, need not be detailed herein.

As discussed herein, “cloud” or “cloud computing” includes a model for enabling convenient, on-demand network access to a shared pool of configurable computing resources (e.g., networks, servers, storage, applications, and services) that can be rapidly provisioned and released with minimal management effort or service provider interaction. Cloud computing may include location-independent computing, whereby shared servers provide resources, software, and data to computers and other devices on demand.

As discussed herein, “transmit” may include sending electronic data from one system component to another over a network connection. Additionally, as used herein, “data” may include encompassing information such as commands, queries, files, data for storage, and the like in digital or any other form.

While certain embodiments of the present disclosure have been described in connection with what is presently considered to be the most practical and various embodiments, it is to be understood that the present disclosure is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the present disclosure and also to enable any person skilled in the art to practice certain embodiments of the present disclosure, including making and using any devices or systems and performing any incorporated methods. The patentable scope of certain embodiments of the present disclosure is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

### EXAMPLES

### Example 1. Machine Learning-Based Method for Optimal Extraction of Bright Features

The detection and quantification of bright features is a common problem in fluorescent microscopy. In applications such as next-generation sequencing (NGS), where maximizing the number of features imaged is crucial, the challenge is exacerbated by feature sizes and optics operating near the diffraction limit. Accurate sequencing relies on accurately quantifying the luminosity of a feature across various wavelength channels, a process known as extraction.

The simplest basecalling approach typically involves selecting the base with the highest intensity, often with an added criterion that the highest intensity must exceed a threshold; otherwise, no base is called. If all intensity values are below the threshold, no call is made. In some cases, the intensity values are normalized by a weighted sum (e.g., to a total of one), and these normalized intensities can be interpreted as probabilities. Before normalization, background signals may be subtracted, and additional factors, such as noise modeled by a Gaussian function, can be incorporated to account for intensity variations. However, this basic approach of selecting the maximum intensity lacks accuracy. For example, when four intensity values (representing the four nucleotide bases) are captured simultaneously, crosstalk can occur between the signals emitted by the fluorophores attached to the respective bases. The signals for each base are detected in distinct channels, each corresponding to a specific wavelength. To mitigate crosstalk and improve accuracy, a crosstalk matrix can be applied, allowing for more accurate intensity values. For instance, the intensity of a specific channel (e.g., the signal for G) at a given position of a nucleic acid can be calculated as a weighted sum of the intensities from all four channels at that same position. However, this correction does not fully account for optical issues or variations in the biochemical process. Another optical issue arises from signal bleed, where the light emitted from neighboring nucleic acids interferes with the measured signal of the nucleic acid being analyzed. This neighboring signal bleed can be addressed through linear or nonlinear regression. For example, the intensity of a specific channel (e.g., the signal for G) at a given position can be calculated as a weighted sum of the intensities from neighboring nucleic acids at the same cycle. A fraction of the neighboring intensities is then subtracted from the measured signal for the nucleic acid of interest. The coefficients for this weighted sum can be determined using optical system measurements. Even with such corrections, this regression approach still faces accuracy limitations, particularly as it does not account for biochemical variations that occur between different experiments.

An approach is to convolve each feature and its immediate neighborhood with a kernel that mimics the expected optical point-spread function (PSF). The PSF is determined by the numerical aperture (NA) of the imaging system and might span, for instance, several pixels. Each convolution extracts the total number of photons emitted by the feature. In this manner, a multi-channel image patch is distilled into a straightforward vector of intensities, with each element corresponding to a different channel. These extracted intensities serve as inputs for basecalling via one of numerous techniques. While this approach can be effective under ideal conditions, effects such as optical aberrations or suboptimal focusing can reduce its efficacy by making the actual feature shapes deviate from that of the kernel.

Here, we present an extraction algorithm that demonstrates enhanced robustness against aberrations and focusing imperfections. We do so by employing a machine learning-based approach that extracts intensity data from multiple kernels and synthesizes the results. We show that this approach not only enhances feature extraction and basecalling accuracy from typical NGS images, but also yields superior results from deliberately misfocused images. Our findings indicate a greater improvement for images with worse focus quality, effectively increasing the usable depth of field. Finally, we discuss the integration of this extraction algorithm into various basecalling frameworks and discuss the potential for future enhancements to its effectiveness and flexibility.

In typical fluorescence-based assays, such as genotyping, spatial sequencings, or traditional next-generation nucleic acid sequencing (whether using real-time, cyclic, or stepwise reaction schemes), dye molecules attached to nucleic acids are excited by an external light source. The subsequent excitation generates fluorescence signals at specific, localized positions on the substrate or in or on a cell, which are captured sequentially over the course of the experiment by an optical system onto an image sensor. These sequential images document the progression of the experiment, capturing each step of the process. For example, in sequencing-by-synthesis (SBS), the extension of a nucleic acid primer along a template is monitored through the incorporation of fluorescently labeled nucleotides, allowing for the determination of the nucleotide sequence. The sequential images are then analyzed to locate the labeled molecules (or clonally amplified clusters) and to quantify the fluorescence signal in terms of wavelength and spatial coordinates. Imaging-based methods enable high-throughput parallelism and multiplexing, significantly reducing the cost and increasing the accessibility of these technologies, with subsequent analysis performed on the collection of images to yield comprehensive results.

The analysis of sequencing images begins with aligning the images to one another across successive cycles and spectral channels with subpixel precision, which can be done via various methods both standard and otherwise. As described herein, the method follows aligning the images to one another across successive cycles and spectral channels. Around each feature, a small image patch of dimension, e.g., 9×9 pixels is defined, allowing features to be analyzed largely independently of one another. Each wavelength channel of an image patch is then convolved with a bank of predefined kernels.

In one example on a patterned substrate (i.e., wherein features align to a sub-pattern, such as a nanofabricated array), the bank comprises four kernels, each constructed based on a different assumed optical NA (either 0.4, 0.6, 0.8, or 1.0). For each NA, a high-resolution Airy disk is created, resulting in a series of Airy disks of different widths. Because we expect our images to comprise a regular, hexagonal grid following the pattern, of bright features, we surround this Airy disk with a hexagonal ring of copies of the opposite sign. The pseudo-inverse of this pattern is then calculated, and is finally downsampled to the true pixel pitch, resulting in a 9×9 kernel that mimics the ideal point spread function with slight depressions for nearest neighbor crosstalk suppression. These implementation details were chosen to match the grid pattern in our anticipated images, but apart from the use of multiple kernels of different widths, they are not fundamental to the function of our invention.

Using the process described above, a different kernel bank is constructed for each channel, corresponding to that channel's predominant wavelength. This bank may be further expanded to include copies with different subpixel translations; for each feature, the appropriate translation will be chosen based on the previously calculated feature location. For the example mentioned, four channels were present, and convolving each with the four kernels results in sixteen extracted scalar intensities.

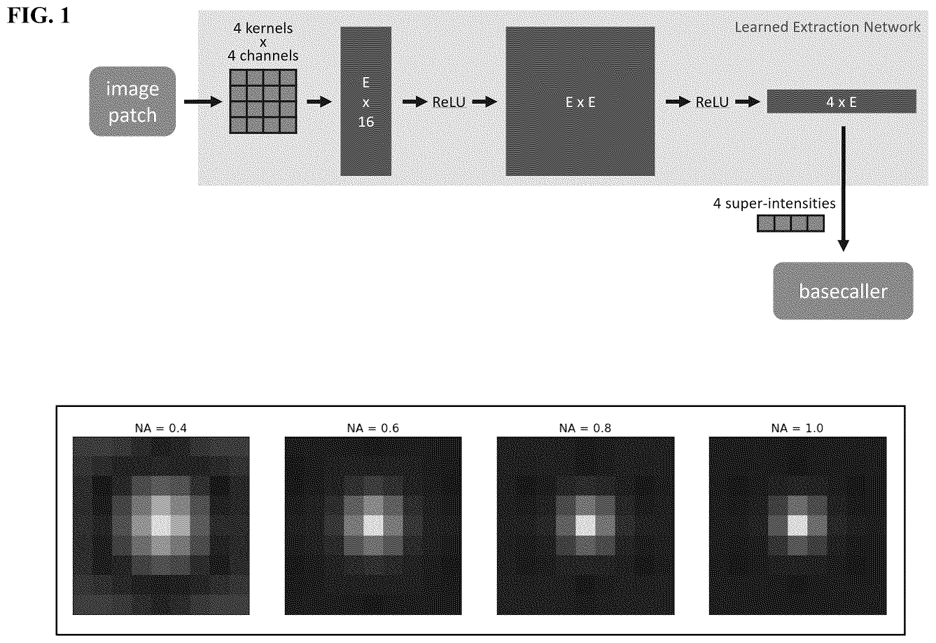

The algorithm uses a machine learning network to compress the extracted intensities into four super-intensities, each of which represents the optimally-extracted signal from a given channel (in this case four spectrally distinct wavelength channels). This allows features that appear larger due to misfocus or other aberrations to still have the majority of their signal extracted by a large kernel, while sharper features can make use of smaller kernels to reduce contamination from background noise or neighboring features. This process is described in FIG. 1. The extracted intensities serve as inputs for a linear transformation, which projects the data into an embedding space of dimension E. The result is passed through a ReLU activation function, another linear transformation into a new, E-dimensional space, another ReLU, and finally a linear transformation down into four dimensions. The result is interpreted as a set of super-intensities and used as the input of a basecaller which is blind to the inner machinations of the learned extraction network. Various basecaller forms known in the art can be used. It is notable, however, that the basecaller is independent of the learned extraction network, both in its structure and in its training: once trained, the learned extraction model can be interposed between an image and a basecaller without altering the latter.

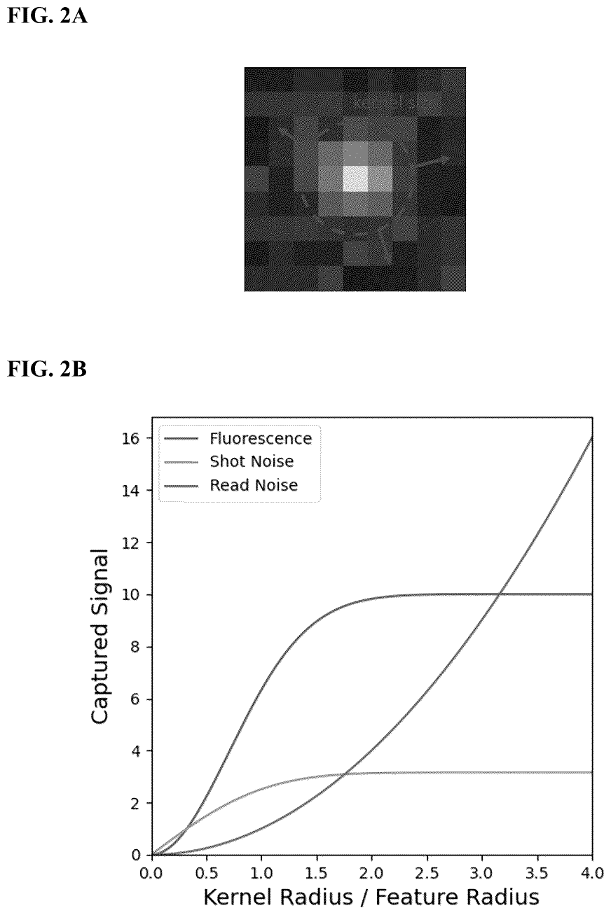

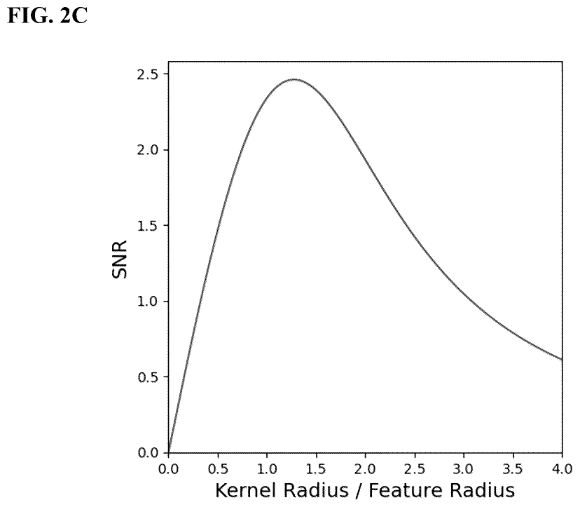

FIGS. 2A-2C illustrates one way of intuiting the role of the learned extraction network. A fluorescent feature takes the form of a bright, local region overlaid on a noisy background. The fidelity with which the feature's intensity can be calculated depends on the signal-to-noise ratio (SNR) between the feature and the background. If we consider extraction based on convolution with a circular kernel of varying radius, we see that each of these terms scales in a different way. As the kernel radius increases, a greater fraction of the signal is contained within it, ultimately saturating once the feature has been fully encircled. As more of the signal is captured, however, the photon shot noise also increases, per its usual Poissonian form. Finally, any camera has some associated read noise, which is uniform from pixel to pixel and therefore scales as the area enclosed by the kernel. By combining these terms, we obtain the SNR curve shown in FIG. 2C, which reaches a maximum at some kernel radius determined by the characteristics of the feature and the background noise. The goal of our extraction network is to use the kernel closest to this optimal point, even as the feature's intensity and focus quality vary.

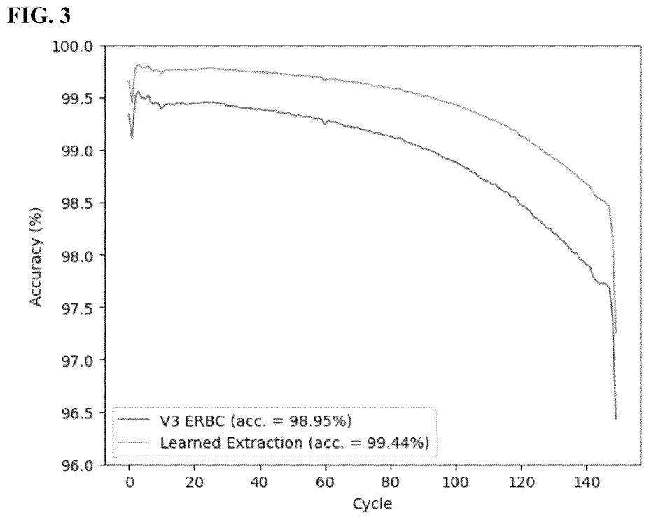

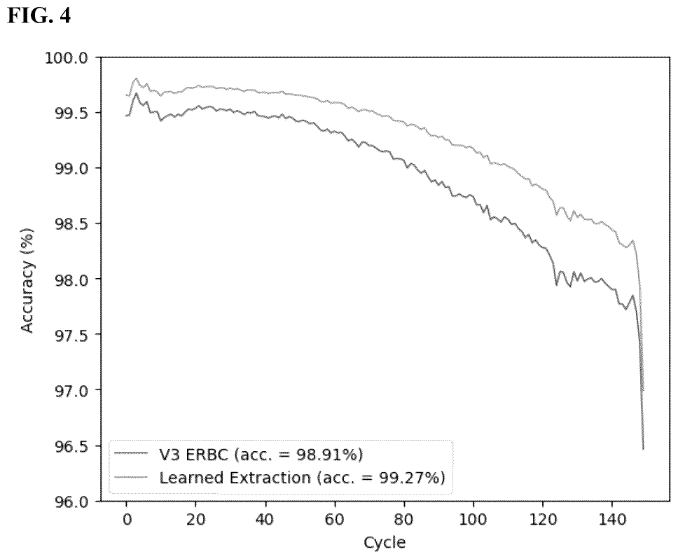

In one example, an embedding dimension of 128 was chosen, and the extraction model was trained on a set of NGS images spanning different imaging systems (in order to cover different optical aberrations) and different levels of misfocus. The trained model was then used to extract feature intensities from a larger set of withheld data, and used as the inputs to a basecaller, referred to here as V3 ERBC. The basecaller did not undergo any additional training. Comparing the basecaller's outputs to the known ground truth showed a significant increase in accuracy when our extraction model was applied to the inputs (FIG. 3).

Another experiment confirmed the generality of our model by applying it to data from an entirely new instrument—i.e., an imaging system it had never been trained on. As in the earlier result, performing the extraction using our model significantly improved the basecaller's accuracy (FIG. 4).

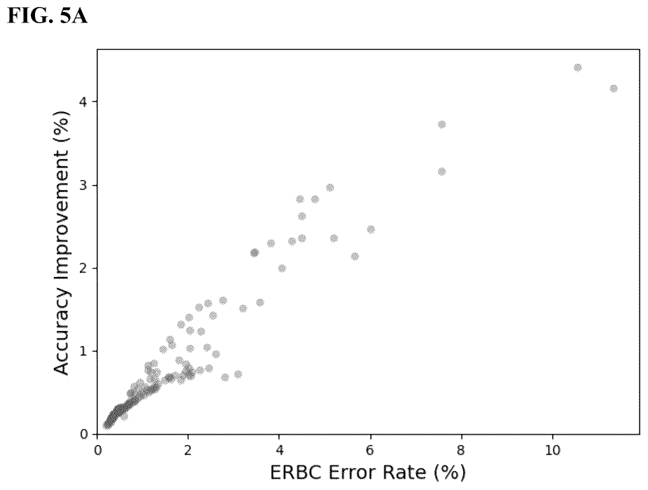

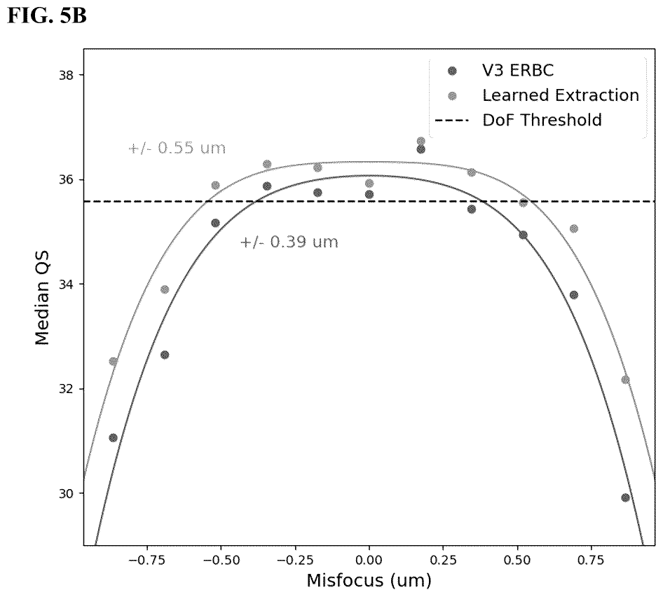

Of particular interest is not only the performance of the basecaller, but also how rapidly it degrades as the images become more defocused i.e., the effective depth-of-focus. To gauge this, we performed an NGS experiment where certain cycles were deliberately defocused by a known amount, and measured the impact on the calculated Phred scores (i.e., Phred score is a measure of the quality of the identification of nucleotides generated by DNA sequencing). The same images were then reanalyzed using our learned extraction model. FIGS. 5A-5B shows that the model preferentially improves the accuracy of cycles that had higher error rates to begin with, resulting in not only an overall increase in accuracy, but a more gradual decrease in performance as a function of misfocus. A typical definition of the depth-of-focus in this system is the point where the median Phred score drops to 0.5 lower than its maximum in-focus value; between the overall elevation and the flattening of this curve, our extraction model increases this by over 40%, resulting in an increased tolerance to misfocus

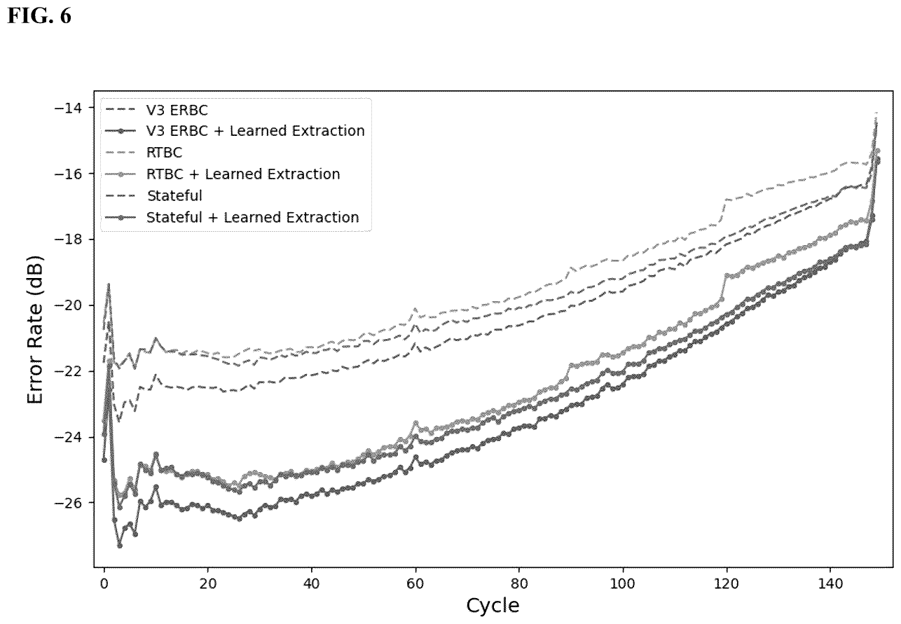

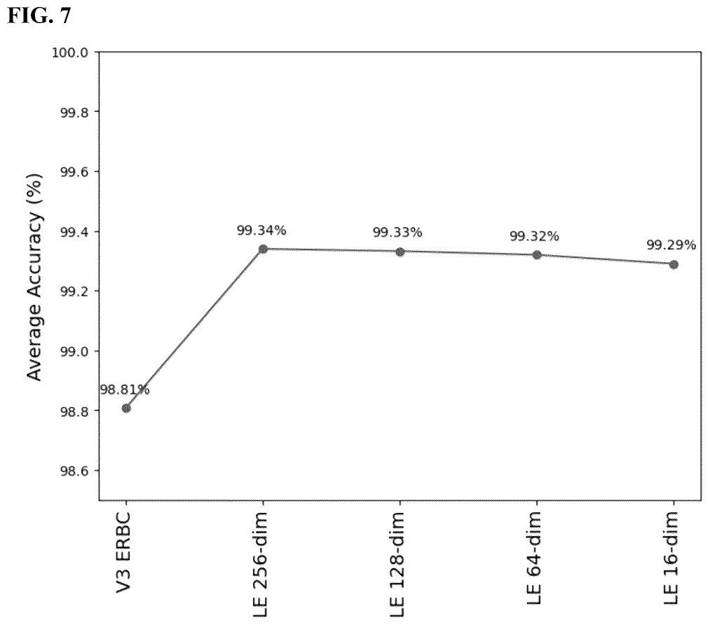

The ability of the extraction approach to calculate optimal feature intensities is not specific to a particular basecaller type. As a demonstration of this, the same extraction network was used to optimize the inputs to three different machine-learning-based basecallers: an end-of-run basecaller (the ERBC mentioned previously), a real-time basecaller (RTBC), and a stateful basecaller, each with different architectural decisions which tradeoff between accuracy, latency, and read/write requirements. All three basecallers had previously been trained on similar datasets without the benefit of the extraction network; in all instances, neither the basecaller nor the extraction model underwent any additional training as a pair. Despite this, passing the inputs first through the extraction model improves the results of all three basecallers by similarly significant amounts (FIG. 6). This suggests that our model is improving the extracted intensities in some generic way, making it a versatile tool for feature extraction.

While the results shown here used a learned extraction network with an embedding dimension of 128, networks of different dimensionality can produce comparable results. FIG. 7 shows the average basecaller accuracy when embedding dimensions of size 256 to 16 are used, with each different network trained independently. Decreasing the dimensionality affords the extraction network fewer opportunities for optimization, but the overall impact on the basecaller accuracy remains minimal, especially compared to the results obtained when no learned extraction model is present (left-most data point). This offers opportunities to trade marginal accuracy improvements for decreases in computational complexity.

In another implementation, the bank of kernels included in the learned extraction network may be modified. For instance, a brute-force search showed that models constructed using only three of the four kernels described above can be trained to be nearly as accurate as the full, four-kernel model, suggesting that the computational complexity can be decreased further without sacrificing performance.

Finally, kernels may also be drawn from beyond the NA-parameterized set explored here. A maximally general model might learn the kernels on a pixel-wise basis as part of the training process; however, this requires optimization over a very large parameter space that may not end up producing physical shapes. A more targeted approach is to project the space of all possible kernels onto some complete basis that mimics the physical realities, such as the Zernike polynomial set which is already frequently used to model optical aberrations. To implement this, a set of Zernike polynomials may be pre-computed, and a kernel bank may be constructed whose elements are learned linear combinations of those polynomials which are trained simultaneously with the rest of the nonlinear extraction network. Once training concludes, the optimal linear combinations can be pre-computed and used in place of the NA-based kernels discussed above without any increase in computational complexity.

What is claimed is:

1. A computer-implemented method for extracting features of sequencing images, comprising:
extracting vector intensities, using at least a machine learning model trained on a set of sequencing images generated from a plurality of imaging systems, of an image patch of the sequencing images; and
compressing, using at least the machine learning model, the vector intensities into a plurality of super intensity vectors.

2. The method of claim 1, further comprising inputting the super intensity vectors into a base caller.

3. The method of claim 1, wherein the sequencing images of the image patch are aligned across successive cycles and spectral channels.

4. The method of claim 1, wherein the image patch is arranged around each extracted feature.

5. The method of claim 1, wherein the plurality of super intensity vector represents optimally-extracted signals of a wavelength channel of the image patch.

6. The method of claim 5, further comprising generating the plurality of super intensity vectors for each wavelength channel by convolving the aligned image patches with a bank of kernels.

7. The method of claim 6, wherein each kernel mimics an expected optical point-spread function.

8. The method of claim 6, further comprising:
constructing a different kernel bank for each wavelength channel, wherein each different kernel bank corresponds to a predominant wavelength of the wavelength channel; and
expanding the different kernel banks to include copies with different subpixel translations based on a previously calculated feature location.

9. The method of claim 6, wherein each of the kernels are constructed based on an assumed optical numerical aperture.

10. The method of claim 9, wherein the assumed optical numerical aperture is at least one of 0.4, 0.6, 0.8, and 1.0.

11. The method of claim 9, further comprising: creating a high-resolution Airy disk for each numerical aperture thereby creating a series of Airy disks of different widths.

12. The method of claim 11, further comprising surrounding each Airy disk by a hexagonal ring.

13. The method of claim 11, further comprising calculating a pseudo-inverse of a pattern created by each Airy disk; and
downsampling the pseudo-inverse to a true pixel pitch, resulting in a kernel that mimics a point spread function with depressions for nearest neighbor crosstalk suppression.

14. The method of claim 1, wherein the compressing the extracted vector intensities comprises:
projecting, using at least a linear transformation, the extracted vector intensities of the image patch into an embedding; and
passing the embedding through a plurality of activation functions and a plurality of linear transformations into the plurality of super intensity vectors.

15. The method of claim 14, further comprising: analyzing, using at least the machine learning model, each extracted feature of the image patch independent of other features of the image patch.

16. A computer-implemented method for extracting features of an image, comprising:
constructing a kernel bank comprising learned linear combinations of a set of precomputed two-dimensional basis vectors trained simultaneously with a machine learning nonlinear extraction network that is trained on a set of sequencing images generated from a plurality of imaging systems; and
pre-computing, using at least the machine learning nonlinear extraction network, a set of optimal linear combinations.

17. A non-transitory computer-readable medium storing instructions that, when executed by a processor, perform a method for analyzing a tissue sample, the method comprising:
extracting vector intensities, using at least a machine learning model trained on a set of sequencing images generated from a plurality of imaging systems, of an image patch of the sequencing images; and
compressing, using at least the machine learning model, the vector intensities into a plurality of super intensity vectors.

18. A system for analyzing a tissue sample, the system comprising:
(a) a memory storing instructions;
(b) a processor configured to execute the instructions to:
extracting vector intensities, using at least a machine learning model trained on a set of sequencing images generated from a plurality of imaging systems, of an image patch of the sequencing images; and
compressing, using at least the machine learning model, the vector intensities into a plurality of super intensity vectors.
